(19) United States
(12) Patent Application Publication
(10) Pub. No.: US 2025/0253623 A1
(43) Pub. Date: Aug. 7, 2025

(54) CIRCULAR HOLE AND NEEDLE BASED AIR CORONA DISINFECTION DEVICE

(71) Applicant: WE AIR HEALTH (SHANGHAI) TECHNOLOGY CO., LTD, Shanghai (CN)

(72) Inventor: Ming ZHANG, Shanghai (CN)

(21) Appl. No.: 19/184,265

(22) Filed: Apr. 21, 2025

Related U.S. Application Data
(63) Continuation of application No. PCT/CN2022/129749, filed on Nov. 4, 2022.

(30) Foreign Application Priority Data
Oct. 25, 2022 (CN) 202222814971.2

(51) Int. Cl. H01T 19/04; A61L 9/22; H01T 23/00

(52) U.S. Cl. CPC H01T 19/04; A61L 9/22; H01T 23/00

(57) ABSTRACT

A circular hole and needle based air corona disinfection device includes: a power supply configured to provide a voltage; a negative plate connected with a negative electrode of the power supply through a wire, and defining a plurality of metal-surrounded circular holes; a discharge alloy needle arranged in each metal-surrounded circular hole; and a positive bracket connected with a positive electrode of the power supply through a wire, in which an end of the discharge alloy needle is fixedly connected inside the positive bracket.

### CROSS-REFERENCE TO RELATED APPLICATION

The present application is a Continuation Application of International Application No. PCT/CN2022/129749, filed on Nov. 4, 2022, which claims the benefit of priority to Chinese Application No. 202222814971.2, filed on Oct. 25, 2022, both of which are incorporated by reference herein in their entireties for all purposes.

### BACKGROUND

After COVID-19, the airborne epidemic virus has become the focus of everyone's attention, the source of infection of most airborne epidemics is the infected patient, and the mode of transmission is that it spreads from person to person when there are people around. Thus, it is particularly important to realize air disinfection in a manned environment.

### SUMMARY

The present disclosure belongs to a field of air disinfection technologies, in particular to a circular hole and needle based air corona disinfection device.

Embodiments of the present disclosure provide a circular hole and needle based air corona disinfection device, which includes a power supply configured to provide a voltage, a negative plate with a metal-surrounded circular hole and a positive bracket.

The negative plate is connected with a negative electrode of the power supply through a wire, a plurality of metal-surrounded circular holes are formed in the negative plate, and a discharge alloy needle is arranged in each metal-surrounded circular hole.

The positive bracket is connected with a positive electrode of the power supply through a wire, one end of the discharge alloy needle is fixedly connected inside the positive bracket, and the other end of the discharge alloy needle is located at a central axis of the metal-surrounded circular hole.

### BRIEF DESCRIPTION OF THE DRAWINGS

FIG. 1 is a schematic view of an overall structure of the present disclosure.

FIG. 2 is an enlarged view of part A in FIG. 1 of the present disclosure.

FIG. 3 is a front view of the present disclosure.

FIG. 4 is an exploded view of the present disclosure.

FIG. 5 is an enlarged view of part B in FIG. 4 of the present disclosure.

FIG. 6 is a sectional view of a negative plate of the present disclosure.

### DETAILED DESCRIPTION

The existing electrostatic precipitator technology mainly relies on adsorption to realize dust removal and purification. A kind of electrostatic technology based on a cylindrical tube and a needle effectively solves the problem that the ozone level exceeds the standard, and uses the corona field generated by a tip of the needle to realize air disinfection, and also, a wall of the tube absorbs the charged particulate matter after passing through the corona zone. However, in the process of realizing the technical scheme in the embodiments of the present disclosure, the inventor of the present disclosure finds that the above technology has at least the following technical problems.

The ozone level of the traditional electrostatic precipitator technology exceeds the standard, and the disinfection ability of the traditional electrostatic precipitator technology is limited. Although the cylindrical tube and needle based technology solves the problem that the ozone level exceeds the standard and produces the corona field to enhance the disinfection ability, the ability of the tube wall to remove dust by absorption is limited. Moreover, the thickness of the whole equipment is also limited, which is generally greater than 2.5 cm, while the traditional filter screen generally has a thickness between 1.5 centimeters and 2.4 centimeters. Thus, this technology cannot directly replace the traditional filter screen (due to lack of installation space).

In a circular hole and needle based air corona disinfection device provided by embodiments of the present disclosure, a cylindrical tube is removed, a circular hole is directly formed in a negative metal plate, and a tip of a discharge alloy needle is used to discharge in the middle of the metal-surrounded circular hole at a central axis of the metal-surrounded circular hole to generate a corona zone, so that the air flowing from the top of the metal-surrounded circular hole to the bottom of the metal-surrounded circular hole is subjected to strong oxidative disinfection.

In order to solve the above problems in the related art, the technical scheme in the embodiments of the present disclosure has the following general idea.

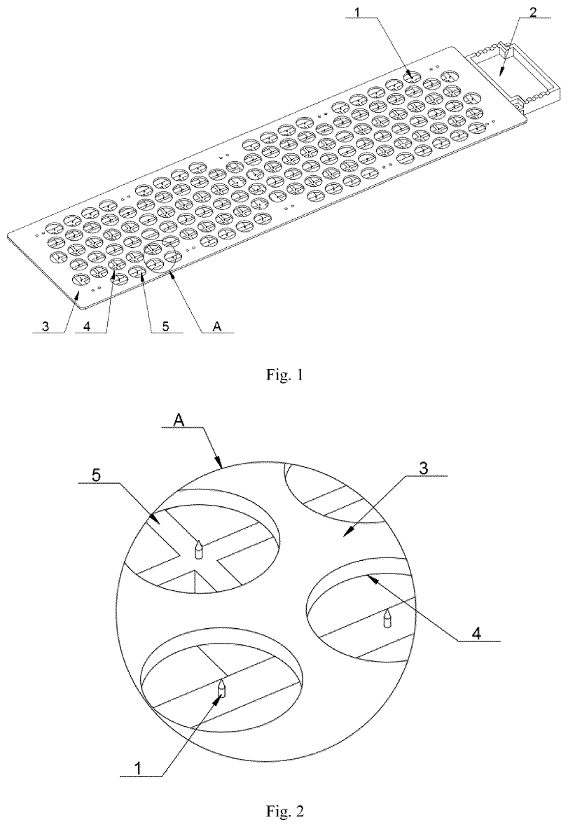

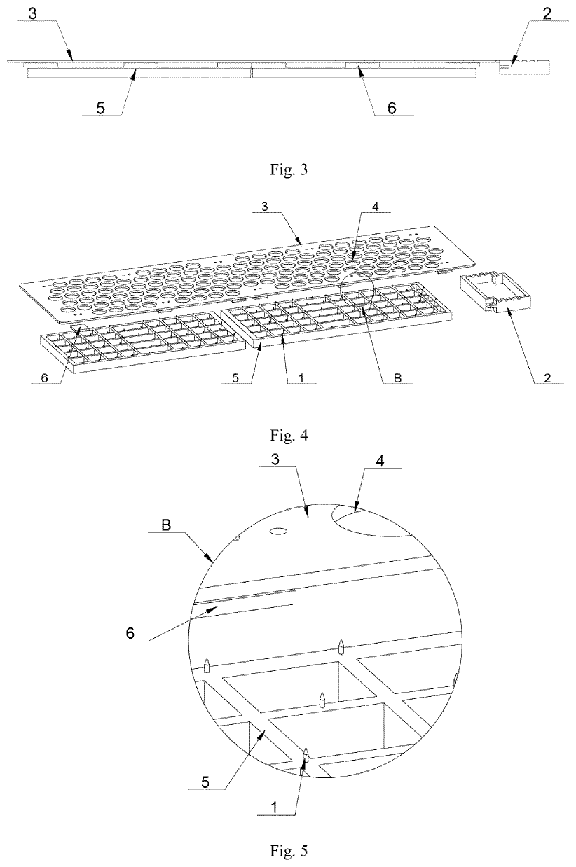

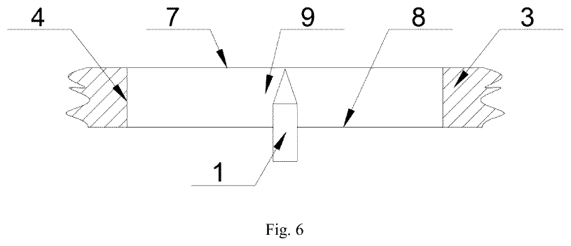

An embodiment of the present disclosure provides a circular hole and needle based air corona disinfection device, as shown in FIGS. 1-6, and the device includes a power supply 2, a negative plate 3, and a positive bracket (e.g., a positive strip) 5.

The power supply 2 is configured to provide a voltage. For example, the power supply 2 may be a high-voltage package configured to provide a relatively high voltage, e.g., a voltage of 3500 volts to 8000 volts.

In order to ensure human safety, the voltage of the power supply 2 is controlled to be between 3500 volts and 8000 volts, and a current of the power supply 2 is controlled to be between 10 microamperes and 1 milliampere, and also is limited at 1 milliampere to ensure the safety of human touch.

The negative plate 3 is connected with a negative electrode of the power supply 2 through a wire, and defines a plurality of metal-surrounded circular holes 4, and a discharge alloy needle 1 is arranged in each metal-surrounded circular hole 4. As described above, the negative plate 3 may be made of metal, so as to define the metal-surrounded circular holes 4.

A tip of the discharge alloy needle 1 discharges generate a large number of free radicals and positive ions, thus exerting a strong oxidative disinfection effect on the air passing through the metal-surrounded circular hole 4.

Further, the metal-surrounded circular hole 4 has an air inlet 7 at a top and an air outlet 8 at a bottom, and the air flows through the metal-surrounded circular hole 4 (i.e., from the air inlet 7 to the air outlet 8) towards the positive bracket 5.

The positive bracket 5 is connected with a positive electrode of the power supply 2 through a wire. One end of the discharge alloy needle 1 is fixed on (for example, fixedly connected inside) the positive bracket 5, and the other end of the discharge alloy needle 1 is located at a central axis of the metal-surrounded circular hole 4. For example, the positive bracket 5 has a shape of a grid, and a plurality of discharge alloy needles 1 may be arranged on the grid, for example, at transverse and longitudinal beams and/or their nodes of the grid.

Further, the other end (i.e., the tip) of the discharge alloy needle 1 is 1-2 millimeters away from the air inlet 7 of the metal-surrounded circular hole 4.

The discharge alloy needle 1 is fitted with the metal-surrounded circular hole 4 formed in the negative plate 3. When the tip of the discharge alloy needle 1 discharges, a confined corona zone 9 which rotates at a high speed is formed in the middle of the metal-surrounded circular hole 4, thus effectively increasing the disinfection path of viruses, bacteria and spore and improves the disinfection efficiency.

For example, charged microorganisms such as viruses, bacteria and spore will generate a spiral motion trajectory under the action of an electromagnetic force in the confined corona zone 9 that rotates at the high speed. This increases the disinfection path of viruses, bacteria and spore, and also increases the collision probability of viruses, bacteria and spore with positive ions generated by the discharge of the discharge alloy needle 1. As a result, the disinfection efficiency is improved.

In addition, the positive ions in the corona zone 9 may move from the tip of the discharge alloy needle 1 to a side wall of the metal-surrounded circular hole 4 under the action of a Coulomb force, so as to diffuse uniformly until they are distributed across an entire cross section of the metal-surrounded circular hole 4 (that is, a section perpendicular to the central axis of the metal-surrounded circular hole 4), thus preventing the local concentration of the positive ions, and expanding a disinfection coverage area. As a result, the disinfection efficiency is further improved.

It may be understood that the high-speed rotation of the corona zone 9 actually refers to the movement of the positive ions in the corona zone 9 along a parabolic curve or a similar curve from the tip of the discharge alloy needle 1 to the side wall of the metal-surrounded circular hole 4. That is, the movement of the positive ions in the corona zone 9 produces a similar effect of the high-speed rotation of the corona zone 9.

In some examples, the central axis of the metal-surrounded circular hole 4 overlaps with an axis of the discharge alloy needle 1 (for example, the discharge alloy needle 1 is oriented upwards vertically), so that the discharge alloy needle 1 is located at the central axis of the metal-surrounded circular hole 4, so as to ensure the uniform discharge of the tip of the needle and limit the generation of ozone, and the discharge alloy needle 1 is fitted with the metal-surrounded circular hole 4 to form the effective corona zone 9 in the middle of the metal-surrounded circular hole 4.

In some examples, the positive bracket 5 is provided with a plurality of insulating connection columns 6 arranged at intervals, and the insulating connection columns 6 are connected between the negative plate 3 and the positive bracket 5, so that the negative plate 3 and the positive bracket 5 remain parallel to each other, and are spaced apart by a distance equal to or larger than 10 millimeters, that is, the interval between the negative plate 3 and the positive bracket 5 is kept equal to or larger than 10 millimeters, thus realizing air insulation.

In some examples, a radius of the metal-surrounded circular hole 4 is 0.7 centimeters to 1.5 centimeters, and a length of the discharge alloy needle is 1.2-1.5 times of the radius of the metal-surrounded circular hole.

In some examples, the voltage of the power supply 2 is denoted as V, the radius of the metal-surrounded circular hole 4 is denoted as R, and V and R satisfy a formula: —1650R²+6250R≤V≤−1650R²+6250R+1500.

It may be understood that the unit of V is volt, and the unit of R is centimeter, while it is specified here that values of R and V satisfy the above formula without considering their units.

In some examples, the radius of the metal-surrounded circular hole 4 is 1.1 centimeters, and the voltage of the power supply 2 is 4878.5 volts to 6378.5 volts, and specifically is 5000 volts to 6000 volts. Further, the current of the power supply 2 is about 500 microamperes.

In this case, when the voltage of the power supply 2 is higher than 6378.5, for example being 6500, the ozone generation will increase significantly, thus failing to satisfy UL 2998 Zero Ozone Emissions Standard for Air Cleaners, i.e. Environmental Claim Validation Procedure (ECVP) for Zero Ozone Emissions from Air Cleaners; and when the voltage of the power supply 2 is less than 4878.5, for example being 4500, the desired disinfection effect cannot be achieved.

In some other examples, the radius of the metal-surrounded circular hole 4 is 0.75 centimeters, and the voltage of the power supply 2 is 3759.4 volts to 5259.4 volts, and specifically is 4000 volts to 4800 volts. Further, the current of the power supply 2 is about 13 microamperes.

In this case, when the voltage of the power supply 2 is higher than 5259.4, for example being 5500, the ozone generation will increase significantly, thus failing to satisfy UL 2998 Zero Ozone Emissions Standard for Air Cleaners, i.e. Environmental Claim Validation Procedure (ECVP) for Zero Ozone Emissions from Air Cleaners; and when the voltage of the power supply 2 is less than 3759.4, for example being 3500, the desired disinfection effect cannot be achieved.

To satisfy the UL 2998 Zero Ozone Emissions Standard for Air Cleaners, the maximum value of ozone emissions at a distance of 2 centimeters from an air outlet of a device within 8 to 24 hours should be less than 5 ppb. Further, details of the UL 2998 Zero Ozone Emissions Standard for Air Cleaners are well known to those skilled in the related art, and hence will not be expatiated here.

In addition, when the radius of the metal-surrounded circular hole 4 is less than 0.7 centimeters, for example being 0.6 centimeters, the disinfection device will not operate normally; and when the radius of the metal-surrounded circular hole 4 is larger than 1.5 centimeters, the voltage of the power supply 2 needs to be increased accordingly, and hence the ozone generation will increase significantly, thus failing to satisfy the UL 2998 Zero Ozone Emissions Standard for Air Cleaners.

Moreover, when the radius of the metal-surrounded circular hole 4 is larger than 1.5 centimeters, even if not considering the ozone generation, it is difficult to generate the confined corona zone, because the positive ions moving along the parabolic curve or the similar curve will form a dead area in the corona zone defined in the metal-surrounded circular hole 4 with the radius larger than 1.5 centimeters. In the dead area, less or even no positive ions exist. Thus, the disinfection effectiveness will be reduced.

Therefore, in the present disclosure, the voltage of the power supply 2 is 3500 volts to 8000 volts, and the radius of the metal-surrounded circular hole 4 is 0.7 centimeters to 1.5 centimeters, thus achieving the desired disinfection effect, e.g. the medical disinfection effect, and satisfying the UL 2998 Zero Ozone Emissions Standard for Air Cleaners.

By adopting the above technical scheme:
in the above design, the tip of the discharge alloy needle 1 is used to discharge in the middle of the metal-surrounded circular hole 4 at the central axis of the metal-surrounded circular hole 4, so that the corona zone 9 may be generated, and thus the air disinfection may be realized in a manned environment. The corona zone 9 which rotates at the high speed is formed at an ionization area in the metal-surrounded circular hole 4 by using the tip of the discharge alloy needle 1 and the high-voltage ionization technology, so that the air flowing from the top of the metal-surrounded circular hole 4 to the bottom of the metal-surrounded circular hole 4 may be subjected to strong oxidative disinfection.

In the process of specific use, the circular hole and needle based air corona disinfection device operates as follows.

In step S1, the power supply 2 with a voltage of 3500-8000 volts and a current of 10 microamperes to 1 milliampere is used to input a current into the discharge alloy needle 1 via the positive bracket 5 through a wire.

In step S2, the tip of the discharge alloy needle 1 discharges to generate a large number of free radicals and positive ions, and form the corona zone 9 which rotates at the high speed in the metal-surrounded circular hole 4, so that the air is subjected to the strong oxidative disinfection.

In step S3, the positive ions and free radicals released by the tip of the discharge alloy needle 1 inside the metal-surrounded circular hole 4 exert the strong oxidative disinfection on the virus flowing through the metal-surrounded circular hole 4, and also, a large number of positive and negative ions will coagulate with the particulate matter, thus effectively reducing PM2.5 in the air.

By adopting the above technical scheme:
in the above design, a large number of free radicals and positive ions are generated by discharging the current from the tip of the discharge alloy needle 1 by means of the power supply 2. When the free radicals and positive ions exert the strong oxidative disinfection on the virus flowing through the metal-surrounded circular hole 4, a large number of positive and negative ions will coagulate with the particulate matter, thus effectively reducing PM2.5 in the air. Therefore, on the basis of inhibiting ozone generation, the corona zone formed by the tip of the alloy needle 1 in the metal-surrounded circular hole 4 is used for efficient air disinfection, and thus the adsorption part of the traditional cylindrical tube is removed, which saves the precious physical space, realizes an ultra-thin structure of 1.5-2 centimeters, facilitates the direct replacement of the traditional filter screen, and also reduces the maintenance work of the cylindrical tube.

In order to overcome the existing shortcomings in the related art, embodiments of the present disclosure provide a circular hole and needle based air corona disinfection device. A cylindrical tube is removed, a circular hole is directly formed in a negative metal plate, and a tip of a discharge alloy needle is used to discharge to generate a corona zone in the middle of the metal-surrounded circular hole at a central axis of the metal-surrounded circular hole, so that the air flowing from the top of the metal-surrounded circular hole to the bottom of the metal-surrounded circular hole is subjected to strong oxidative disinfection, which solves the problems that the ozone level in the existing electrostatic precipitator technology exceeds the standard and the disinfection capacity of the existing electrostatic precipitator technology is limited, and also solves the problems that the circular hole and needle based electrostatic technology has a large thickness and the maintenance of the cylindrical tube is troublesome.

The technical scheme adopted by the embodiments of the present disclosure to solve the technical problems is as follows.

A circular hole and needle based air corona disinfection device includes a power supply configured to provide a voltage, a negative plate with a metal-surrounded circular hole and a positive bracket.

The negative plate is connected with a negative electrode of the power supply through a wire, a plurality of metal-surrounded circular holes are formed in the negative plate, and a discharge alloy needle is arranged in each metal-surrounded circular hole.

The positive bracket is connected with a positive electrode of the power supply through a wire, one end of the discharge alloy needle is fixedly connected inside the positive bracket, and the other end of the discharge alloy needle is located at a central axis of the metal-surrounded circular hole.

In a possible implementation, a voltage of the power supply is controlled to be between 3500 volts and 8000 volts, and a current of the power supply is controlled to be between 10 microamperes and 1 milliampere.

In a possible implementation, the central axis of the metal-surrounded circular hole overlaps with an axis of the discharge alloy needle.

In a possible implementation, the discharge alloy needle is located at the central axis of the metal-surrounded circular hole.

In a possible implementation, the positive bracket is in a grid shape.

In a possible implementation, the positive bracket is provided with a plurality of insulating connection columns arranged at intervals, and the negative plate is fixedly connected with the positive bracket through the plurality of insulating connection columns.

In a possible implementation, the negative plate and the positive bracket remain parallel to each other and are spaced apart by a distance equal to or larger than 10 millimeters.

In a possible implementation, a distance between a tip of the discharge alloy needle and an air inlet of the metal-surrounded circular hole is 1-2 millimeters.

In a possible implementation, an interior of the metal-surrounded circular hole is divided into an air inlet at a top of the metal-surrounded circular hole, a corona zone in the middle of the metal-surrounded circular hole and an air outlet at a bottom of the metal-surrounded circular hole.

In a possible implementation, the discharge alloy needle is fitted with the metal-surrounded circular hole in the negative plate, so that the corona zone is formed in the middle of the metal-surrounded circular hole in a case that a tip of the discharge alloy needle discharges.

In a possible implementation, a radius of the metal-surrounded circular hole is 0.7 centimeters to 1.5 centimeters, and a length of the discharge alloy needle is 1.2-1.5 times of the radius of the metal-surrounded circular hole.

In a possible implementation, the voltage of the power supply is denoted as V, the radius of the metal-surrounded circular hole is denoted as R, and V and R satisfy a formula: −1650R²+6250R≤V≤−1650R²+6250R+1500.

In a possible implementation, the radius of the metal-surrounded circular hole is 1.1 centimeters, and the voltage of the power supply is 4878.5 volts to 6378.5 volts.

In a possible implementation, the voltage of the power supply is 5000 volts to 6000 volts.

In a possible implementation, the radius of the metal-surrounded circular hole is 0.75 centimeters, and the voltage of the power supply is 3759.4 volts to 5259.4 volts.

In a possible implementation, the voltage of the power supply is 4000 volts to 4800 volts.

The present disclosure has the following beneficial effects.

First, in this scheme, the tip of the discharge alloy needle is used to discharge to generate the corona zone in the middle of the metal-surrounded circular hole at the central axis of the metal-surrounded circular hole, so that the corona field exerts strong oxidative disinfection on the air flowing from the top of the metal-surrounded circular hole to the bottom of the metal-surrounded circular hole.

Second, in this scheme, by using free radicals and positive ions released from the tip of the discharge alloy needle, a large number of positive and negative ions will coagulate with the particulate matter, thus effectively reducing the PM2.5 in the air, and the adsorption part of the traditional cylindrical tube is removed, thus saving the precious physical space, facilitating the direct replacement of the traditional filter screen, and reducing the difficulty of maintaining the cylindrical tube in the later stage.

Finally, it should be noted that obviously, the above embodiments are only examples for clearly explaining the present disclosure, and they are not limitations to the implementations. For those skilled in the art, other changes or modifications in different forms may be made on the basis of the above description. It is not necessary and impossible to exhaust all the embodiments here. However, the obvious changes or modifications derived therefrom are still within the protection scope of the present disclosure.

What is claimed is:

1. A circular hole and needle based air corona disinfection device, comprising:
a power supply configured to provide a voltage;
a negative plate connected with a negative electrode of the power supply through a wire, and defining a plurality of metal-surrounded circular holes;
a discharge alloy needle arranged in each metal-surrounded circular hole; and
a positive bracket connected with a positive electrode of the power supply through a wire, wherein an end of the discharge alloy needle is fixed to the positive bracket.

2. The circular hole and needle based air corona disinfection device according to claim 1, wherein a voltage of the power supply is controlled to be between 3500 volts and 8000 volts, and a current of the power supply is controlled to be between 10 microamperes and 1 milliampere.

3. The circular hole and needle based air corona disinfection device according to claim 1, wherein a central axis of the metal-surrounded circular hole overlaps with an axis of the discharge alloy needle.

4. The circular hole and needle based air corona disinfection device according to claim 3, wherein the discharge alloy needle is located at the central axis of the metal-surrounded circular hole.

5. The circular hole and needle based air corona disinfection device according to claim 1, wherein the positive bracket is in a grid shape.

6. The circular hole and needle based air corona disinfection device according to claim 1, wherein the positive bracket is provided with a plurality of insulating connection columns arranged at intervals, and the negative plate is fixedly connected with the positive bracket through the plurality of insulating connection columns.

7. The circular hole and needle based air corona disinfection device according to claim 6, wherein the negative plate and the positive bracket remain parallel to each other and are spaced apart by a distance equal to or larger than 10 millimeters.

8. The circular hole and needle based air corona disinfection device according to claim 1, wherein a distance between a tip of the discharge alloy needle and an air inlet of the negative plate is 1-2 millimeters.

9. The circular hole and needle based air corona disinfection device according to claim 1, wherein an interior of the metal-surrounded circular hole is divided into an air inlet at a top of the metal-surrounded circular hole, a corona zone in the middle of the metal-surrounded circular hole and an air outlet at a bottom of the metal-surrounded circular hole.

10. The circular hole and needle based air corona disinfection device according to claim 9, wherein the discharge alloy needle is fitted with the metal-surrounded circular hole in the negative plate, so that the corona zone is formed in the middle of the metal-surrounded circular hole in a case that a tip of the discharge alloy needle discharges.

11. The circular hole and needle based air corona disinfection device according to claim 1, wherein a radius of the metal-surrounded circular hole is 0.7 centimeters to 1.5 centimeters, and a length of the discharge alloy needle is 1.2-1.5 times of the radius of the metal-surrounded circular hole.

12. The circular hole and needle based air corona disinfection device according to claim 11, wherein the voltage of the power supply is denoted as V, the radius of the metal-surrounded circular hole is denoted as R, and V and R satisfy a formula: −1650R²+6250R≤V≤−1650R²+6250R+1500.

13. The circular hole and needle based air corona disinfection device according to claim 12, wherein the radius of the metal-surrounded circular hole is 1.1 centimeters, and the voltage of the power supply is 4878.5 volts to 6378.5 volts.

14. The circular hole and needle based air corona disinfection device according to claim 13, wherein the voltage of the power supply is 5000 volts to 6000 volts.

15. The circular hole and needle based air corona disinfection device according to claim 12, wherein the radius of the metal-surrounded circular hole is 0.75 centimeters, and the voltage of the power supply is 3759.4 volts to 5259.4 volts.

16. The circular hole and needle based air corona disinfection device according to claim 15, wherein the voltage of the power supply is 4000 volts to 4800 volts.
